(19) United States
(12) Patent Application Publication
(10) Pub. No.: US 2006/0015132 A1
(43) Pub. Date: Jan. 19, 2006

(54) Probe introducer with valve assembly to minimize air entry

(76) Inventors: Robert F. Rioux, Ashland, MA (US); Kristian Dimatteo, Waltham, MA (US)

Correspondence Address: BINGHAM, MCCUTCHEN LLP, THREE EMBARCADERO CENTER, 18 FLOOR, SAN FRANCISCO, CA, 94111-4067, US

(21) Appl. No.: 10/892,866

(22) Filed: Jul. 16, 2004

(51) Int. Cl. A61M 29/00

(52) U.S. Cl. 606/191

(57) ABSTRACT

A medical probe introducer includes a cannula having an axial lumen, and a valve assembly connected to the cannula. The valve assembly is configured to allow passage of a medical probe (e.g., a stylet probe, a biopsy probe, or a RF ablation probe), while substantially preventing the passage of air, through the cannula lumen.

### FIELD OF INVENTION

The present invention relates generally to devices for introducing medical probes into the body, e.g., for performing a tissue biopsy or a radio frequency (RF) ablation procedures.

### BACKGROUND OF THE INVENTION

Medical probes are slender, flexible instruments designed for introduction into a organ, cavity, or solid tissue in the body for purposes of exploration or treatment. The use of a probe allows for minimally invasive procedures to be completed with (typically) fewer complications than procedures using open surgery. While probes may also be used in open surgery, one of the advantages of using a probe is its adaptability for percutaneous procedures.

For example, medical probes are particularly suited for procedures in the lungs. In order to introduce a working medical probe into a lung, a solid core stylet is positioned in an inner lumen of an introducer cannula, which is then passed percutaneously through the patient's chest wall, until the distal tip of the stylet and distal cannula opening are positioned at a target location in the lung. The stylet is then withdrawn from, and the working probe inserted into, the cannula lumen. As the stylet and cannula pass through the pleural cavity, it is possible for air to enter into the cavity from between the outside diameter of the stylet and the inside diameter of the cannula. Similarly, during removal of the stylet and introduction of a working probe in the cannula lumen, the same possibility of air passage into the pleural cavity from between the outer diameter of the probe and the inner diameter of the cannula exists. Once air enters the pleural cavity, pressure in the pleura can become greater than the pressure in the lung, causing the lung to partially or completely collapse.

Thus, it is desirable to provide an apparatus for introducing a medical probe into a lung, or other body organ or cavity, while minimizing the unwanted and/or dangerous ancillary entry of air into the body.

### SUMMARY OF THE INVENTION

In accordance with the present inventions, an introducer for accessing an internal body region is provided. The introducer comprises a cannula having an axial lumen configured to receive a medical probe and a distal port from which the medical probe can be deployed. In one embodiment, the cannula is rigid, although in some cases, the cannula may be semi-rigid or even flexible. The introducer further comprises a valve assembly located on cannula in communication with the distal cannula port. The valve assembly is preferably located at the proximal end of the cannula in order to minimize the design constraints on the valve assembly, although the valve assembly can be located anywhere along the cannula without straying from the principles taught by this invention.

In accordance with one aspect of the inventive introducer, the valve assembly is configured to allow passage of the medical probe, while substantially sealing the distal cannula port from an external environment during passage of the medical probe. In accordance with another separate aspect of the inventive introducer, the valve assembly comprises a slit valve in communication with the cannula lumen. The slit valve is configured to allow passage of the medical probe. In accordance with still another separate aspect of the inventive introducer, the valve assembly is a multi-stage valve assembly to further ensure that the distal cannula port is sealed. The multi-stage valve assembly comprises a first valve configured to allow passage of the medical probe, while substantially sealing the distal cannula port from an external environment during passage of the medical probe, and a manually adjustable second valve, such as a rotary valve, configured to seal around the medical probe. In one embodiment, the first valve also seals the cannula lumen from the external environment when the medical probe does not reside within the valve assembly.

In accordance with the present inventions, a medical kit comprising any one of the previous introducers is provided. The medical kit further comprises a medical probe, such as a stylet probe, therapeutic probe (e.g., a radio frequency ablation probe), or a diagnostic probe (e.g., a biopsy probe). Optionally, the medical kit comprises two or more medical probes that can be exchanged within the introducer.

In accordance with the present inventions, a method of accessing an internal region (e.g., a lung) within a body is provided. The method comprises introducing a cannula into the body (e.g., using a percutaneous procedure), wherein a distal port of the cannula is adjacent the internal region (e.g., in the pleural cavity if the internal region is the lung), and introducing a medical probe (such as a stylet probe, diagnostic probe, or therapeutic probe) through the cannula to deploy the medical probe from the distal cannula port. The cannula may be introduced into the body while the medical probe is deployed from the distal cannula port, in which case, the medical probe will be used to pierce tissue. The method may optionally comprise using the medical probe to perform a medical procedure (e.g., a therapeutic or diagnostic procedure) on the internal region.

In accordance with one aspect, the inventive method further comprises substantially sealing the distal cannula port from an external environment while the medical probe is introduced through the cannula. The distal cannula port can be sealed at a location proximal to the cannula, although it can be sealed anywhere along the cannula as well without straying from the principles taught by this invention. The distal cannula port can optionally also be substantially sealed prior to introducing the medical probe through the cannula. The inventive method may also comprise removing the medical probe from the cannula, in which case, the method will further comprise substantially sealing the distal cannula port from the external environment while the medical probe is removed from the cannula.

In accordance with another aspect, the method further comprises removing the medical probe from the cannula, and introducing another medical probe to deploy the other medical probe from distal cannula port. In this case, the distal cannula port is substantially sealed from the external environment while both medical probes are introduced into the cannula and while the first medical probe is removed from the cannula.

Other aspects and features of the invention will be evident from the following detailed description of the illustrated embodiments, which are provided to illustrate, and not to limit, the invention.

### BRIEF DESCRIPTION OF THE DRAWINGS

The drawings illustrate the design and utility of preferred embodiment(s) of the invention, in which similar elements are referred to by common reference numerals. In order to better appreciate the advantages and objects of the invention, reference should be made to the accompanying drawings that illustrate the preferred embodiment(s). The drawings, however, are not drawn to scale and depict only some embodiment(s) of the invention, and therefore, should not be taken as limiting its scope. With this caveat, the embodiment(s) of the invention will be described and explained with additional specificity and detail through the use of the accompanying drawings in which:

FIG. 1 is a side view of an exemplary medical probe introducer constructed in accordance with one embodiment of the invention, wherein a stylet probe is particularly shown received within the introducer;

FIG. 2 is a side view of the introducer of FIG. 1, wherein a radio frequency (RF) ablation probe is particularly shown received within the introducer;

FIG. 3 is a side view of the introducer of FIG. 1, wherein a biopsy probe is particularly shown received within the introducer;

FIG. 4 is a partially cutaway perspective view of a valve assembly used in the introducer of FIG. 1;

FIG. 5 is a partially cutaway perspective view of the valve assembly of FIG. 4, particularly showing a stylet probe introduced through the valve assembly;

FIG. 6 is a partially cutaway-perspective view of a modification of the valve assembly of FIG. 4;

FIG. 7 is a partially cutaway perspective view of a dual-stage valve assembly that can alternatively be used in the introducer of FIG. 1;

FIG. 8 is a partially cutaway perspective view of a modification of the dual-stage valve assembly of FIG. 7;

FIG. 9 is a partially cutaway perspective view of the dual-stage valve assembly of FIG. 7, particularly showing a stylet probe introduced through the valve assembly;

FIG. 10 is a partially cutaway perspective view of another dual-stage valve assembly that can alternatively be used in the introducer of FIG. 1;

FIG. 11 is a partially cutaway perspective view of a modification of the dual-stage valve assembly of FIG. 10;

FIG. 12 is a partially cutaway perspective view of the dual-stage valve assembly of FIG. 10, particularly showing a stylet probe introduced through the valve assembly; and

FIGS. 13A-13D are side views illustrating a method of using the introducer, stylet probe, RF ablation probe, and biopsy probe of FIGS. 1-3 to percutaneously treat a tumor within a lung of a patient.

### DETAILED DESCRIPTION OF THE ILLUSTRATED EMBODIMENTS

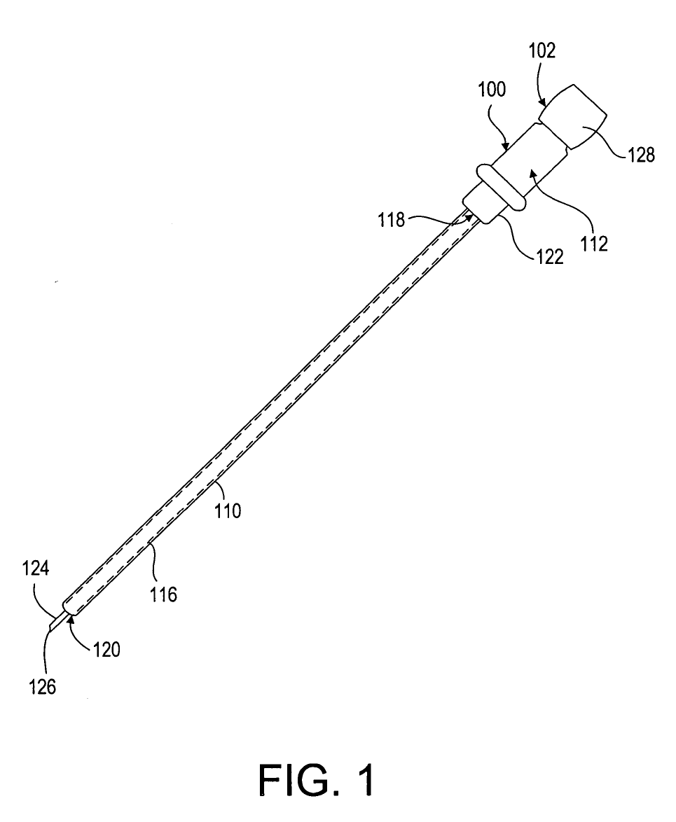

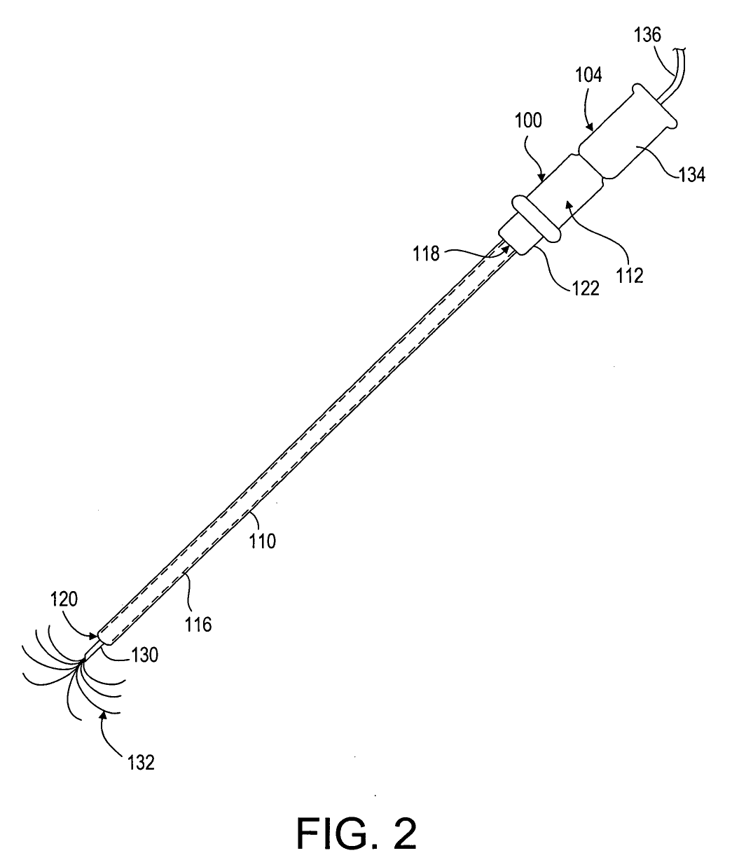

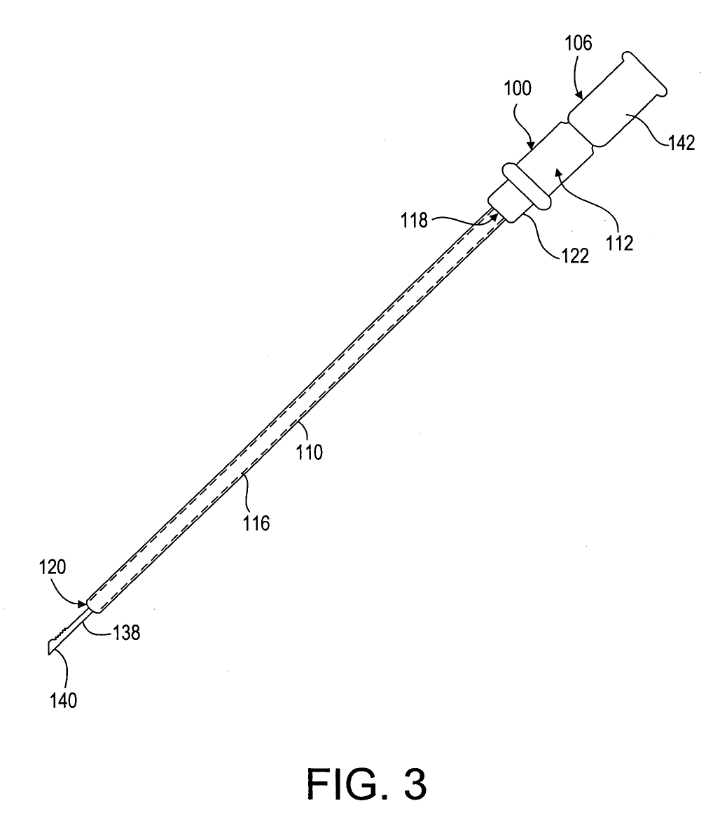

FIGS. 1-3 illustrate an exemplary introducer 100 that can be used with a variety of medical probes, including a solid core stylet probe 102 (FIG. 1), a radio frequency (RF) ablation probe 104 (FIG. 2), and a biopsy probe 106 (FIG. 3). Together, the introducer 100, stylet probe 102, ablation probe 104, and biopsy probe 106 can be packaged and sold as a kit to a medical care provider, such as hospital. As will be described in further detail below, a physician may advance the introducer 100 into a patient's body, and place the stylet probe 102, ablation probe 104, and biopsy probe 106 into contact with an internal region within the patient's body. These medical probes can be exchanged in and out of the introducer 100 without exposing the internal body region to the environment external to the patient's body.

To this end, the introducer 100 generally comprises a cannula 110 configured to be percutaneously introduced into a patient's body, and a valve assembly 112 configured to provide a seal that prevents the internal body region from being exposed to the external environment during introduction of the cannula 110 and subsequent exchange of the stylet probe 102, ablation probe 104, and biopsy probe 106. In this embodiment, the valve assembly 112 is conveniently located at the proximal end of the cannula 110, so that it resides outside of the patient's body when the cannula 110 is inserted into the patient's body. Alternatively, however, the valve assembly 112 can be located anywhere along the cannula 110—although such placement will not be as convenient, since additional design constraints would need to be imposed on the valve assembly 112, and in particular, the profile of the valve assembly 112 would have to match that of the cannula 110.

The cannula 110 comprises a cannula shaft 114 that is preferably rigid or semi-rigid, and constructed from medical grade metal or plastic. The cannula shaft 114 is generally a hollow tube having an axial lumen 116 (shown in phantom) extending from a proximal end opening 118 to a distal end opening 120. The lumen 116 is sized to allow the probes (i.e., the stylet probe 102, ablation probe 104, and biopsy probe 106) to be alternately positioned therein, e.g., with the diameter of the lumen 116 preferably being 0.001 inch to 0.020 inch greater than the outer diameter of the shafts of the probes. The cannula 110 further comprises a proximal connector 122 mounted on the proximal end of the cannula shaft 114 for mating with the valve assembly 112, as will be described in further detail below.

As illustrated in FIG. 1, the stylet probe 102 comprises a shaft 124 that is preferably rigid or semi-rigid, and constructed from medical grade metal or plastic. The stylet probe 102 has a tissue piercing tip 126 located at the distal end of the shaft 124 for penetrating through a patient's skin and underlying tissue and/or organ(s), while minimizing tissue trauma. That is, the stylet tip 124 prevents the cannula 110 from coring tissue when introduced through the tissue. The length of the stylet shaft 124 is selected, such that the stylet tip 126 deploys from the distal cannula opening 120 a predetermined distance when the stylet probe 102 is fully engaged with the cannula 110, as illustrated in FIG. 1. The stylet probe 102 further comprises a proximal connector 128 mounted on the proximal end of the stylet shaft 124 for mating with the valve assembly 112, as will be described in further detail below. The proximal connector 128 is shaped to act as a handle for grasping by a physician when advancing the stylet probe 102 through the introducer 100.

As illustrated in FIG. 2, the RF ablation probe 104 comprises a shaft 130 that is preferably rigid or semi-rigid, and constructed from medical grade metal or plastic. The ablation probe 104 comprises an electrode element 132 (shown here as an electrode array) mounted to the distal end of the probe shaft 130 for therapeutically ablating targeted tissue. The length of the ablation probe shaft 130 is selected, such that the electrode element 132 deploys from the distal cannula opening 120 a predetermined distance when the ablation probe 104 is fully engaged with the cannula 110, as illustrated in FIG. 2. The ablation probe 104 further comprises a proximal connector 134 mounted on the proximal end of the probe shaft 130 for mating with the valve assembly 112, as will be described in further detail below. The proximal connector 134 is shaped to act as a handle for grasping by a physician when advancing the ablation probe 104 through the introducer 100. The proximal connector 134 also comprises a RF electrical connector (not shown) that allows the ablation probe 104 to be mated within an RF generator via a RF cable 136.

In alternative embodiments, the ablation probe 104 may have a solid core shaft with a sharpened tip or may otherwise be designed to be self-penetrating and prevent tissue coring. In this case, the use of the stylet probe 102 may not be needed, and the cannula 110 can be introduced through tissue with the ablation probe 104 in place.

As illustrated in FIG. 3, the biopsy probe 106 comprises a shaft 138 that is preferably rigid or semi-rigid, and constructed from medical grade metal or plastic. The biopsy probe 106 further comprises a biopsy element 140 (shown here as a slicing mechanism) mounted to the distal end of the probe shaft 138 for removing tissue for diagnostic purposes. The length of the probe shaft 138 is selected, such that the biopsy element 140 deploys from the distal cannula opening 120 a predetermined distance when the biopsy probe 106 is fully engaged with the cannula 110, as illustrated in FIG. 3. The biopsy probe 106 further comprises a proximal connector 142 mounted on the proximal end of the probe shaft 138 for mating with the valve assembly 112, as will be described in further detail below. The proximal connector 142 is shaped to act as a handle for grasping by a physician when advancing the biopsy probe 106 through the introducer 100.

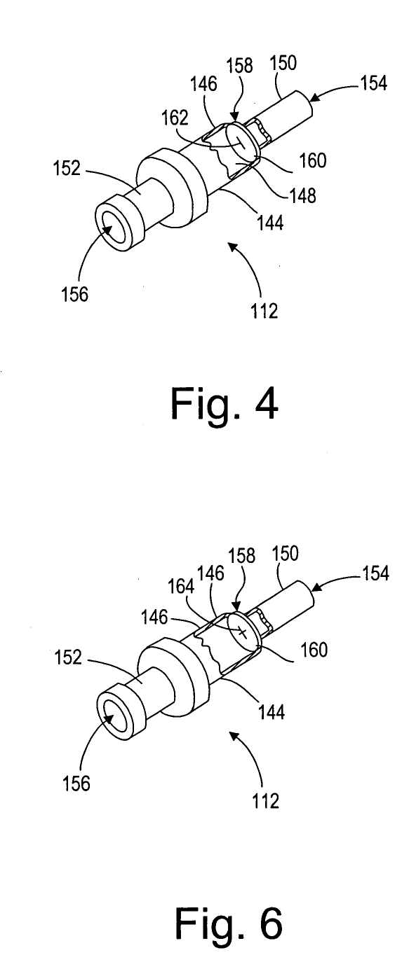

Referring additionally to FIG. 4, the valve assembly 112 of the cannula 110 comprises a valve housing 144 having a generally annular wall 146. The housing wall 146 has a uniform thickness and has a cylindrical exterior surface and an interior surface, which defines a lumen 148 that extends the length of the valve housing 144. When the valve assembly 112 is mated with the cannula 110, as is shown in FIGS. 1-3, the housing lumen 146 is axially aligned and in communication with the cannula lumen 116 to allow passage of the probes through the respective valve assembly 112 and into the cannula 110.

The valve housing 144 of the valve assembly 112 comprises a proximal connector 150 that defines a proximal opening 154 into which a probe can be inserted and passed into the valve lumen 148, and a distal connector 150 that forms a distal opening 154 from which the probe can exit into the cannula lumen 116. The proximal connector 150 is configured to mate with the respective connectors 128, 134, and 142 of the stylet probe 102, ablation probe 104, and biopsy probe 106, and the distal connector 152 is configured to mate with the cannula connector 122. In the illustrated embodiment, the stylet, ablation probe, and biopsy probe connectors 128/134/142 and the cannula connector 122 are female luer connectors, in which case, the proximal and distal valve connectors 150/152 are male luer connectors. Alternatively, any or all of the stylet, ablation probe, and biopsy probe connectors 128/134/142 and the cannula connector 122 can be male luer connectors, in which case, the respective proximal valve connector 150 and/or distal valve connector 152 will accordingly be female luer connectors. It should be appreciated that any mating set of connectors capable of joining with a complementary fitting and providing an integral fit between the probe and valve housing 144, as well as creating an air tight seal between the distal valve opening 152 and the cannula lumen 116, is suitable for use.

The valve housing 144 can be constructed of any material that provides durability and rigidity, such as a molded medical grade synthetic resinous material or plastic. In the illustrated embodiment, the valve housing 144 is formed of a unibody structure, but may be constructed as separately formed components that are subsequently integrated with each other, e.g., by bonding. For example, the proximal and distal valve connectors 150/152 can be separately formed, and then bonded to the respective ends of the valve housing 144.

The valve assembly 112 may comprise any one of a variety of sealing mechanisms that allow passage of the selected probe through the valve lumen 148 and into the cannula lumen 116, while sealing the cannula lumen 116, and thus the distal cannula opening 120, from the external environment. In particular, the sealing mechanism prevents air from entering the valve lumen 148, and being conveyed through the cannula lumen 116 where it can escape out of the distal cannula opening 120 into the patient's body.

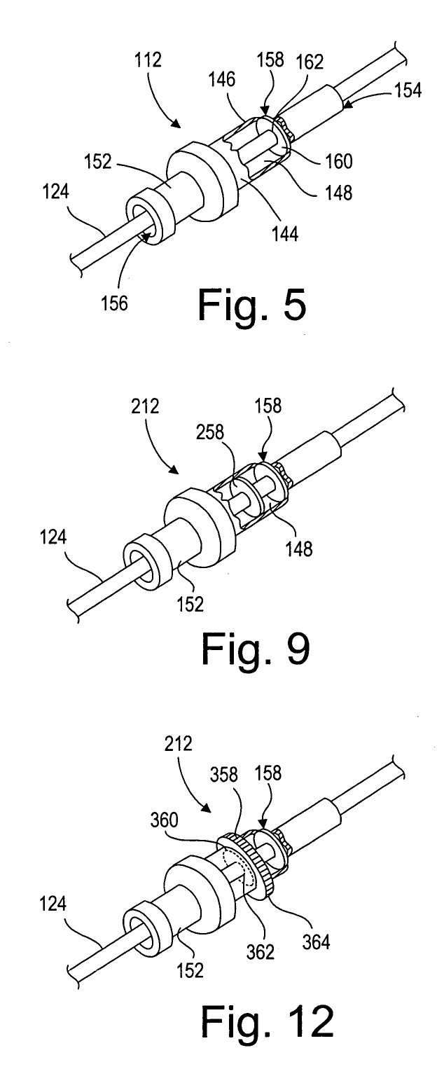

For example, as shown in FIG. 4, the valve assembly 112 comprises a slit valve 158 having a thin membrane 160 and a slit 162 formed within the center of the membrane 160. The membrane 160 is preferably made from silicone or other medical grade material that has flexibility, resilience, and tensile strength over a wide temperature range. The membrane 160 is contained within the proximal end of the valve housing 144 and extends transversely across the valve lumen 148 to seal it from the external environment. The edges of the membrane 160 may be retained within an annular recess (not shown) formed within the valve housing 144. Due to the resilient nature of the slit 162, it is designed to sealingly close when a probe does not reside within the valve lumen 148. The size of the slit 162 is designed to accommodate the shafts of the probes. For example, as illustrated in FIG. 5, the slit 162 transforms into a circular opening that conforms to the outer circumference of a probe shaft (in this case, the stylet shaft 124) to seal it as the probe shaft is introduced therethrough. Thus, it can be appreciated that the slit valve 158 seals the cannula lumen 116, and thus, the distal cannula opening 120, from the external environment when a probe is both inserted into the valve lumen 148 and not inserted into the valve lumen 148. Further details regarding the manufacture and use of slit valves are disclosed in U.S. Pat. No. 5,843,044, which is expressly incorporated herein by reference.

In the embodiment illustrated in FIG. 4, the slit valve 158 has a single slit 162 that is formed through the membrane 160. Alternatively, as shown in FIG. 6, a pair of intersecting slits 164 are provided to allow for a greater variety of probe sizes to be used with the valve assembly 112. In particular, the slit pair 164 provides for greater flexibility by allowing the opening in the membrane 160 to vary in size over a greater range, while still surrounding a probe along its outer circumference. While this embodiment illustrates slits 164 as being orthogonal to each other to form a cross, this is not intended to be a limitation on the design of the valve assembly 112. The angle between the two slits 164 may vary and the lengths of the slits may vary as well, depending on the size of the probes that are intended to fit through the valve assembly 112. In addition, a greater number of slits can be used to provide the valve assembly 112 with more flexibility.

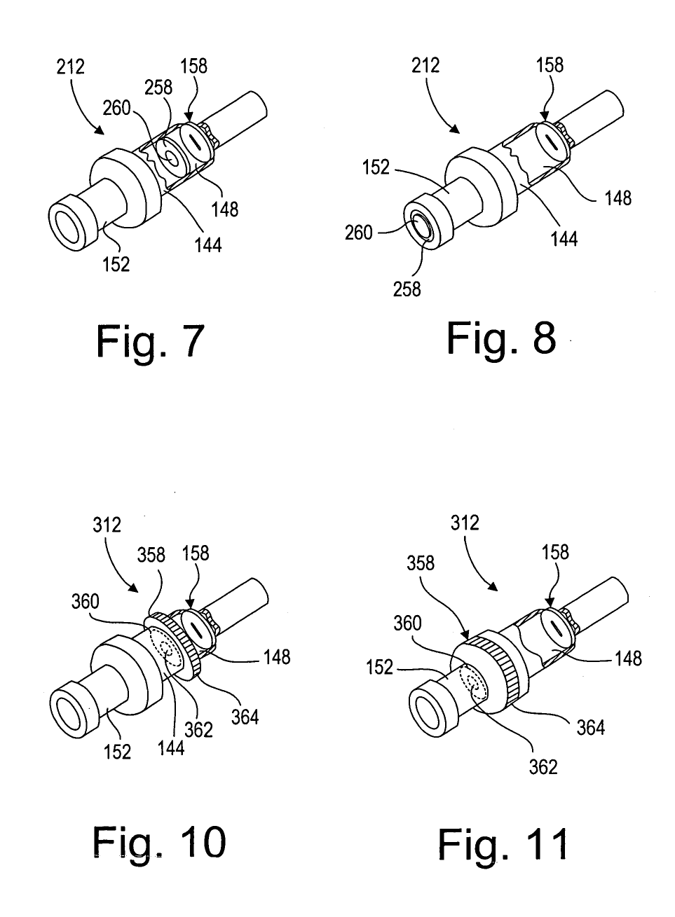

FIG. 7 illustrates another embodiment of a valve assembly 212 that can alternatively be used in the introducer 100 of FIG. 1. The valve assembly 212 is similar to the previously described valve assembly 112, with the exception that it is a two-stage valve assembly that additionally comprises a flexible ring 258, such as a grommet. The flexible ring 258 may be composed of rubber, plastic or any other nonporous flexible material suitable for medical applications that is capable of acting as a seal with a probe. The flexible ring 258 is positioned within the valve housing 144 distal to the slit valve 158 and extends transversely across the valve lumen 148. The edges of the flexible ring 258 may be retained within an annular recess (not shown) formed within the valve housing 144. Alternatively, as shown in FIG. 8, the flexible ring 258 can be positioned within the distal connector 152. In either embodiment, the flexible ring 258 comprises a permanent opening 260 designed to tightly receive and seal a probe shaft (in this case, the stylet shaft 124) when placed through valve lumen 148, as shown in FIG. 9, thereby providing an additional mechanism for sealing the cannula lumen 116, and thus distal cannula opening 120, from the external environment. As previously discussed, when a probe is not inserted through the valve lumen 148, the slit valve 158 will seal the cannula lumen 116 and distal cannula opening 120 from the external environment. Alternatively, one or more slits (not shown) can radially extend outward from the opening 260 to facilitate insertion of the probe shaft through the valve lumen 148.

FIG. 10 illustrates another embodiment of a valve assembly 312 that can alternatively be used in the introducer 100 of FIG. 1. The valve assembly 312 is similar to the previously described valve assembly 112, with the exception that it is a two-stage valve assembly that additionally comprises a manually adjustable valve 358 associated with the valve housing 144. In the embodiment illustrated in FIG. 10, the adjustable valve 358 is a rotary valve, and in particular, a Touhy-Borst valve, that comprises a flexible washer 360 with an opening 362 (shown in phantom). The flexible washer 360 may be composed of rubber, plastic or any other nonporous flexible material suitable for medical applications that is capable of acting as a seal with a probe. The flexible washer 258 is positioned within the valve housing 144 distal to the slit valve 158 and extends transversely across the valve lumen 148. The edges of the flexible ring 258 may be retained within an annular recess (not shown) formed within the valve housing 144. Alternatively, as shown in FIG. 11, the flexible washer 360 (shown in phantom) is located within the lumen of the distal connector 152.

The adjustable valve 358 further comprises a compression nut 362 coupled to the outside of the valve housing 144 (in the case of the FIG. 10 embodiment) or the distal connector 152 (in the case of the FIG. 11 embodiment). The nut 362 can be rotated to compress the washer 360, thereby modifying the size of the opening 362. As a result, the opening 362 of the flexible washer 360 can be adjusted to tightly receive any one of a variety of differently sized probe shafts, as illustrated in FIG. 12. Thus, the adjustable valve 358 provides an additional mechanism for sealing the cannula lumen 116, and thus distal cannula opening 120, from the external environment. As previously discussed, when a probe is not inserted through the valve lumen 148, the slit valve 158 will seal the cannula lumen 116 and distal cannula opening 120 from the external environment.

It should be noted that the valve combinations disclosed in these embodiments are intended to be exemplary only and are not intended to be a limitation on the design of any particular valve or valve assembly for use in embodiments of the invention. Any combination of valves that work cooperatively to prevent fluid or air flow in the cannula lumen 104 when a device is inserted or removed could be incorporated into this design. In addition, the aforementioned valve assemblies have been described as being separate units that can be mounted to the cannula 110 to form the introducer 100. Alternatively, these valve assemblies can be fabricated with the cannula 110 to form a unibody introducer 100.

Having described the detailed structure of the various embodiments of the invention, a kit including the introducer 100, stylet probe 102, ablation probe 104, and biopsy probe 106, will now be described in performing a medical procedure on a tissue region within a patient's body. The tissue region may be located anywhere in the body where hyperthermic exposure may be beneficial. Most commonly, the tissue region will comprise a solid tumor within an organ of the body, such as the lung, liver, kidney, pancreas, breast, prostrate (not accessed via the urethra), and the like. The use of the kit lends itself particularly well in the treatment of lung tumors where the threat of Pneumothorax is great. The volume to be treated will depend on the size of the tumor or other lesion, typically having a total volume from 1 cm³ to 150 cm³, and often from 2 cm³ to 35 cm³. The peripheral dimensions of the tissue region may be regular, e.g., spherical or ellipsoidal, but will more usually be irregular. The tissue region may be identified using conventional imaging techniques capable of elucidating a target tissue, e.g., tumor tissue, such as ultrasonic scanning, magnetic resonance imaging (MRI), computer-assisted tomography (CAT), fluoroscopy, nuclear scanning (using radiolabeled tumor-specific probes), and the like. Preferred is the use of high resolution ultrasound of the tumor or other lesion being treated, either intraoperatively or externally.

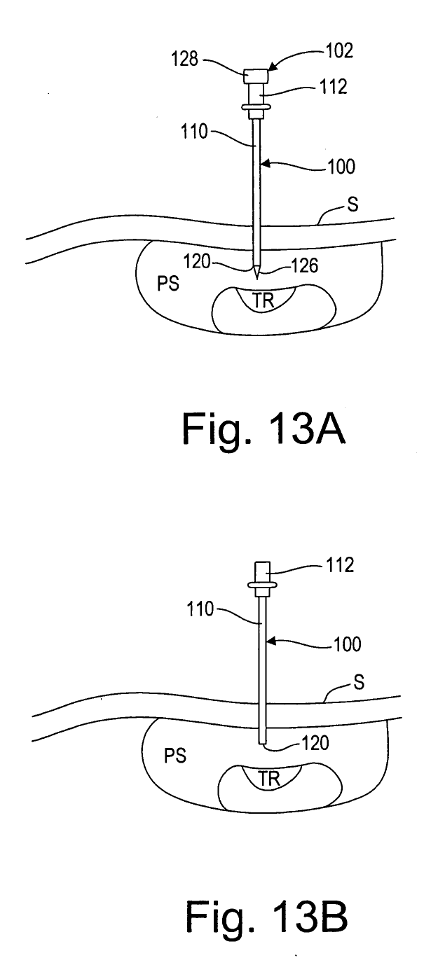

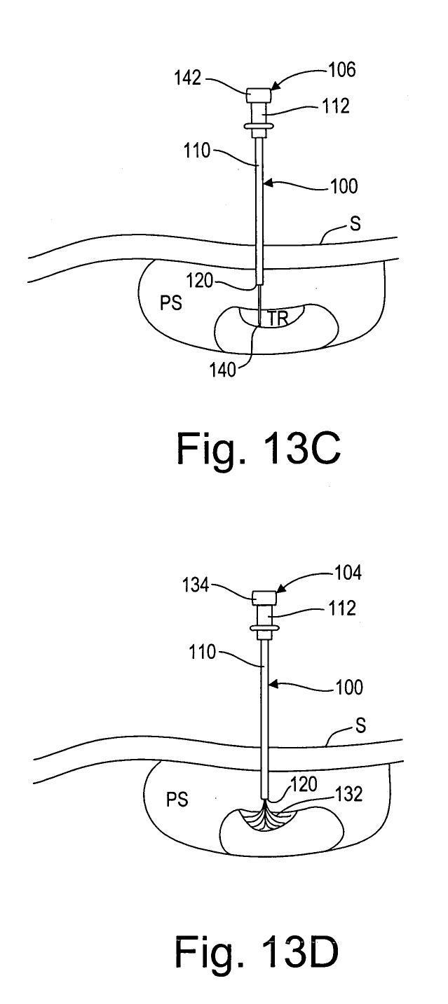

Referring now to FIGS. 13A-13D, the treatment of a tissue region TR located beneath the skin S of a patient will now be described. In this method, the tissue region TR is a tumor that has formed on the lung of the patient. First, the introducer 100 is assembled by connecting the valve assembly 112 to the cannula 110, and specifically, by mating the distal valve connector 152 to the proximal cannula connector 112. Optionally, the valve assembly 112 may be part of a preassembled co-access cannula set or the valve assembly 112 may form an integral portion of the cannula 110, in which case, the valve assembly 112 need not be separately connected to the cannula 110.

Once the introducer 100 is assembled, the stylet probe 102 is inserted through the valve assembly 112 into the cannula lumen 116 until the distal stylet tip 126 deploys from the distal cannula opening 120 and the stylet connector 128 mates with the proximal valve connector 150, as illustrated in FIG. 1. At this point, the valve mechanism of the valve assembly 112, and in particular, the slit valve 158, seals around the stylet shaft 124, thereby sealing the cannula lumen 116 and distal cannula opening 120 from the external environment. If the dual-stage valve assembly 212 is alternatively used, the additional valve mechanism, and in particular, the flexible ring 258, will also seal around the stylet shaft 124. If the dual-stage valve assembly 312 is alternatively used, the compression nut 364 on the valve 358 can be adjusted to tightened the flexible washer 360 onto the stylet shaft 124 after the stylet probe 102 has been inserted into the introducer 100.

Next, the introducer 100, with the stylet probe 102, is positioned at the region of the patient where access will be provided to the tissue region. In this method, the tissue region is in the lung, and therefore the access point will be in the chest region of the patient. The introducer 100, facilitated by the stylet tip 126, is then percutaneously advanced through the patients skin S and chest wall until the distal cannula opening 120 is adjacent the tissue region TR within the pleural space PS of the patient (FIG. 13A). Alternatively, if the introducer 100 has a non-coring tip and is rigid enough, it can be percutaneously advanced into the patient without the use of the stylet probe 102. In either case, because the valve assembly 112 seals the distal cannula opening 120 from the external environment, no air will leak from the external environment through the cannula lumen 116 and into the pleural space PC, thereby preventing the onset of pneumothorax.

Once the introducer 100 is in place, the stylet probe 102 is removed from the introducer 100 (FIG. 13B). Because the slit valve 158 of the valve assembly 112 continuously seals around the stylet shaft 124 while it is being removed from the introducer 100, the distal cannula opening 120 remains sealed from the external environment, thereby preventing air from leaking through the cannula lumen 116 and into the pleural space PS. If the dual-stage valve assembly 212 is alternatively used, the flexible ring 258, will also seal around the stylet shaft 124 as it is removed from the introducer. If the dual-stage valve assembly 312 is alternatively used, the compression nut 364 on the valve 358 will be adjusted to loosen the flexible washer 360 on the stylet shaft 124 prior to its removal from the introducer 100.

The biopsy probe 106 is then inserted through the valve assembly 112 into the cannula lumen 116 until the biopsy mechanism 140 deploys from the distal cannula opening 120 into the tissue region TR and the probe connector 142 mates with the proximal valve connector 150 (FIG. 13C). Because the slit valve 158 of the valve assembly 112 continuously seals around the probe shaft 138 while it is being advanced into the introducer 100, the distal cannula opening 120 remains sealed from the external environment, thereby preventing air from leaking through the cannula lumen 116 and into the pleural space PS. If the dual-stage valve assembly 212 is alternatively used, the flexible ring 258, will also seal around the probe shaft 138 as it is advanced into the introducer 100. If the dual-stage valve assembly 312 is alternatively used, the compression nut 364 on the valve 358 will be adjusted to tighten the flexible washer 360 on the probe shaft 138 after it has been fully advanced into the introducer 100.

With a sample tissue region captured by the biopsy mechanism 140, the biopsy probe 106 is removed from the introducer 100. Again, the distal cannula opening 120 is sealed from the environment during removal of the biopsy probe 106 in the same manner that it was sealed during removal of the stylet probe 102. The ablation probe 106 is then inserted through the valve assembly 112 into the cannula lumen 116 until the electrode element 132 deploys from the distal cannula opening 120 into the tissue region TR and the probe connector 134 mates with the proximal valve connector 150 (FIG. 13D). The distal cannula opening 120 is sealed from the environment during introduction of the ablation probe 104 in the same manner that it was sealed during introduction of the biopsy probe 106. The ablation probe 104 is then connected to an RF generator (not shown) and operated to therapeutically ablate the tissue region TR, after which the introducer 100, with the ablation probe 104, is removed from the patient.

Optionally, prior to removing the introducer 100 from the patient, the ablation probe 104 can be removed from the introducer 100 while the distal cannula opening 120 is sealed by the valve mechanism 112, so that a biocompatible sealing agent can be delivered through the valve assembly 112, through the cannula lumen 116, and out of the distal cannula opening 120 as the introducer 100 is being removed from the patient. In this manner, the access channel previously created by the introducer 100 can be sealed to prevent the entry of air into the pleural cavity PS when the introducer 100 is removed from the channel.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

1. An introducer for accessing an internal body region, comprising:
a cannula having an axial lumen configured to receive a medical probe and a distal opening from which the medical probe can be deployed; and
a valve assembly located on the cannula in communication with the distal cannula opening, the valve assembly configured to allow passage of the medical probe, while substantially sealing the distal cannula opening from an external environment during-passage of the medical probe.

2. The introducer of claim 1, wherein the valve assembly comprises a slit valve.

3. The introducer of claim 1, wherein the valve assembly is a multi-stage valve assembly.

4. The introducer of claim 3, wherein the multi-stage valve assembly comprises a slit valve and a grommet.

5. The introducer of claim 3, wherein the multi-stage valve assembly comprises a slit valve and a rotary valve.

6. The introducer of claim 1, wherein the valve assembly is located proximal to the cannula lumen, and is configured to allow passage of the medical probe into the cannula lumen.

7. The introducer of claim 1, wherein the cannula is rigid.

8. A medical kit, comprising the introducer of claim 1 and the medical probe.

9. The medical kit of claim 8, wherein the medical probe is one of a stylet probe, a therapeutic probe, and a diagnostic probe.

10. The medical kit of claim 8, further comprising another medical probe, wherein the cannula lumen is configured to receive the other medical probe, and the valve assembly is configured to allow passage of the other medical probe, while substantially sealing the distal cannula opening from the external environment during passage of the other medical probe.

11. An introducer for accessing an internal body region, comprising:
a cannula having an axial lumen configured to receive a medical probe; and
a slit valve in communication with the cannula lumen, the slit valve configured to allow passage of the medical probe.

12. The introducer of claim 11, further comprising a rotary valve in communication with the cannula lumen.

13. The introducer of claim 11, wherein the slit valve is located proximal to the cannula lumen, and is configured to allow passage of the medical probe into the cannula lumen.

14. The introducer of claim 11, wherein the cannula is rigid.

15. A medical kit, comprising the introducer of claim 11 and the medical probe.

16. The medical assembly of claim 15, wherein the medical probe is one of a stylet probe, a therapeutic probe, and a diagnostic probe.

17. The medical assembly of claim 15, further comprising another medical probe, wherein the cannula lumen is configured to receive the other medical probe, and the slit valve is configured to allow passage of the other medical probe.

18. An introducer for accessing an internal body region, comprising:
a cannula having an axial lumen configured to receive a medical probe, and a distal opening from which the medical probe can be deployed; and
a multi-stage valve assembly located on the cannula in communication with the cannula lumen, the valve assembly comprising a first valve configured to allow passage of the medical probe, while substantially sealing the distal cannula opening from an external environment during passage of the medical probe, and a manually adjustable second valve configured to seal around the medical probe.

19. The introducer of claim 18, wherein the first valve is a slit valve.

20. The introducer of claim 18, wherein the second valve is a rotary valve.

21. The introducer of claim 18, wherein the first valve seals the cannula lumen from the external environment when the medical probe does not reside within the valve assembly.

22. The introducer of claim 18, wherein the first valve is located proximal to the cannula lumen, and is configured to allow passage of the medical probe into the cannula lumen.

23. The introducer of claim 18, wherein the cannula is rigid.

24. A medical kit, comprising the introducer of claim 18 and the medical probe.

25. The medical kit of claim 24, wherein the medical probe is one of a stylet probe, a therapeutic probe, and a diagnostic probe.

26. The medical kit of claim 24, further comprising another medical probe, wherein the cannula lumen is configured to receive the other medical probe, the first valve is configured to allow passage of the other medical probe, while substantially sealing the distal cannula opening from the external environment during passage of the other medical probe, and the second valve is configured to seal around the other medical probe.

27.-43. (canceled)
